(19) United States
(12) Patent Application Publication
(10) Pub. No.: US 2017/0328889 A1
(43) Pub. Date: Nov. 16, 2017

(54) SYSTEMS AND METHODS OF DISEASE MODELING USING STATIC AND TIME-DEPENDENT HYDROGELS

(71) Applicant: The Regents of the University of California, Oakland, CA (US)

(72) Inventor: Adam Engler, San Diego, CA (US)

(21) Appl. No.: 15/531,915

(22) PCT Filed: Dec. 4, 2015
(86) PCT No.: PCT/US15/64025

§ 371 Date: May 31, 2017

Related U.S. Application Data
(60) Provisional application No. 62/088,405, filed on Dec. 5, 2014.

(51) Int. Cl. G01N 33/50

(52) U.S. Cl. CPC G01N 33/5044; G01N 33/5011; G01N 2800/325

(57) ABSTRACT

Provided are methods and devices for the selection and regulation of the mechanical properties of substrates or tissue microenvironments as a technique to model disease progression in tissues. Substrate mechanical properties include elasticity, which is varied dynamically. Also provided are methods and devices for screening for compounds useful for treating such diseases.

### CROSS REFERENCE TO RELATED APPLICATION(S)

This application claims the benefit of priority under 35 U.S.C. §119(e) of U.S. Ser. No. 62/088,405, filed Dec. 5, 2015, the entire content of which is incorporated herein by reference.

### GRANT INFORMATION

This invention was made with government support under Grant Nos. DP02OD006460 and R21HL106529 awarded by The National Institutes of Health. The United States government has certain rights in the invention.

### BACKGROUND OF THE INVENTION

### Field of the Invention

The invention relates generally to hydrogels and more specifically to altering the properties of certain hydrogels to mimic environmental changes of diseased tissue.

### Background Information

Identification and evaluation of new therapeutic agents or identification of suspected disease associated targets typically employ animal models which are expensive, time consuming, require skilled animal-trained staff and utilize large numbers of animals. In vitro alternatives have relied on the use of conventional cell culture systems which are limited in that they do not allow the three-dimensional interactions that occur between cells and their surrounding tissue.

Normal tissue cells are generally not viable when suspended in a fluid. Thus, they are “anchorage-dependent” because to grow, such cells must adhere to a solid matrix, varying in stiffness from rigid glass to soft agar, topography, and thickness (e.g., basement membrane). Anchorage-dependent cells, therefore, are no longer viable if dissociated from the solid matrix and suspended in the culture media, even if soluble proteins are added to engage cell adhesion molecules, e.g., integrin-binding RGD peptide.

Fluids are clearly mechanically distinct from solids, which flow when stressed, whereas solids have the ability to resist sustained deformation. In most soft tissues—skin, muscle, brain, etc.—adherent cells together with an extracellular matrix constitute a relatively elastic microenvironment. Macroscopically, elasticity (measured as ‘Pascal’ or newtons/square meters) is evident in the ability of a solid tissue to resist deformation, e.g., mild poking or pinching or even after sustained compression, and return to its original shape. The degree or amount of deformation in a given tissue for the same amount of force changes from one tissue to the next. The softer a tissue is, the less force is require to deform it the same amount. Differences in tissue level elasticity arise naturally, making brain (<10³ Pa) softer than muscle (˜10⁴ Pa), which is softer than de-mineralized bone (3×10⁴ Pa) (Engler et al, Cell 2006; Discher et al, Science 2009). At the cellular scale, normal tissue cells probe elasticity as they adhere and pull on their surroundings. Such processes are dependent in part on myosin-based contractility and transcellular adhesions—centered on integrins, cadherins, and perhaps other adhesion molecules—to transmit forces to substrates. Consequently, adhesion complexes and the actomyosin cytoskeleton, whose contractile forces are transmitted through transcellular structures, play key roles in molecular pathways.

Microenvironments and niches appear important in stem cell lineage specification and differentiation as cells can ‘feel’ tissue softness via contractile forces, generated by cross-bridging interactions of actin and myosin filaments. These forces (referred to as traction forces) are transmitted to the substrate, causing wrinkles or strains in thin films or soft gels (Harris et al., Science 208:177 (1980); Oliver et al., J. Cell Biol. 145:589 (1999); Marganski et al., Methods Enzymol. 361:197 (2003); Balaban et al., Nat. Cell Biol. 3:466 (2001); Tan et al., Proc. Natl. Acad. Sci. USA 100:1484 (2003)). The cell, in turn, responds to the resistance of the substrate by adjusting its adhesions, cytoskeleton, and overall state, e.g., differentiation. Thus, cells not only sense and respond to chemical cues, they also respond to the stiffness or flexibility of the tissue around them, collectively called the extracellular matrix (ECM). ECM stiffness can determine whether a cell proliferates or stays quiescent.

For example, adult stem cells, as part of normal regenerative processes, are believed to migrate or circulate and engraft to sites of injury, and will differentiate within these various in vivo microenvironments, ranging from compliant tissue substrates, such as brain or muscle, to rigid tissue substrates, such as bone. Mesenchymal stem cells (MSCs) are pluripotent, anchorage-dependent, and bone marrow-derived cells differentiating into various types of anchorage-dependent cells, including neurons, myoblasts, osteoblasts, and more (Gang et al., Stem Cells 22:617-624 (2004); Gilbert et al., J. Biol. Chem. 277, 2695-2701 (2002); McBeath et al., Developmental Cell 6: 483-495 (2004); Pittenger et al., Science 284:143-147 (1999); Salim et al., J. Biol. Chem. 279:40007-40016 (2004); Tanaka et al., J. Cell Biochem. 93, 454-462 (2004)) via different signaling paths. Soluble factors and cell density clearly influence these differentiation pathways chemically, but variations can also be physical (Gregory et al., Science STKE PE37 (2005); Salasznyk et al., J. Biomed. Biotechnol. 24-34 (2004)). For instance, stem cells adhere and differentiate in soft brain tissue or near rigid bone, and in vitro on soft gels or hard plastic culture dishes. However, compounding MSC-based therapies which consider physical matrix effects are normal wound healing responses, where the formation of fibrotic scar tissue will stiffen the microenvironment, and genetic disorders, such as muscular dystrophy, which increase fibrosis in affected tissues (Engler et al., 2004c, supra).

This wide range in substrate stiffness between tissue types can be altered by disease. For example after a heart attack or “myocardial infarction (MI),” heart muscle becomes devoid of oxygen, i.e., hypoxic, and the muscle dies. The heart then remodels itself to continue its pumping function, but with reduced efficiency. Remodeling includes the programmed cell death, i.e., apoptosis, of muscle and replacement with connective tissues including extracellular matrix (ECM). However this process results in the formation non-contractile and more rigid tissue <5×10⁴ Pa (Berry et al, AJP: Heart Circ Physiol 2006). At the cellular level, these changes strongly influence focal adhesions and cytoskeletal assembly (Beningo et al., J. Cell Biol. 153:881-888 (2001); Bershadshy et al., Annu. Rev. Cell Dev. Biol. 19:677-695 (2003); Discher et al., Science, 310:1139-1143 (Nov. 18, 2005); Engler et al., Biophys. J. 86:617-628 (2004a); Engler et al., J. Cell Biol. 166: 877-887 (2004c); Georges et al., J. Appl. Physiol. 98:1547-1553 (2005); Pelham et al., Proc. Natl. Acad. Sci. USA 94:13661-13665 (1997); Yeung et al., Cell Motil. Cytoskeleton 60:24-34 (2005)) and are modulated by Rho GTPases and their effectors (Gregory et al., 2005, supra; Paszek et al., Cancer Cell 8:241-254 (2005); Peyton et al., J. Cell Physiol. 204:198-209 (2005)).

Efforts to build biosynthetic materials or engineered tissues that recapitulate these structure-function relationships often fail because of the inability to replicate the in vivo conditions that coax this behavior from ensembles of cells. Thus, a need exists for rapid, low-cost devices to screen therapeutic agents to treat diseases in the appropriate disease-specific context.

### SUMMARY OF THE INVENTION

Methods are provided to create and mimic normal and pathological tissues on a chip using hydrogels and cross-linkers to create and manipulate the structure and stiffness of the physical environment for the cells. In vitro induction of altered phenotypes for heart and mammary epithelial cells when the hydrogel is crosslinked to resemble the stiffness of diseased tissues, e.g., myocardial infarction and breast cancer, respectively, are demonstrated herein. Accordingly, the methods described herein allow disease modeling, both dynamically and statically sampling, as well as the ability to maintain constant culture stiffness at any point during the pathological processes.

In one aspect, the invention provides a method of mimicking progression of human breast cancer in a hydrogel. The method includes providing a methacrylated hyaluronic acid (MeHA) hydrogel having an elasticity defined by elastic constant E, wherein the MeHA hydrogel comprises a photoinitiator such as Irgacure 2959; exposing the MeHA hydrogel to UV radiation and for sufficient time to crosslink the hydrogel so as to achieve an elasticity of about 100 Pascal (Pa); seeding the MeHA hydrogel with an anchorage-dependent cell and allowing the cell to differentiate into a committed cell type; and thereafter exposing the MeHA hydrogel to additional photoinitiator and additional UV radiation. In various embodiments, the MeHA hydrogel is overlayed with Matrigel after seeding and prior to differentiating. In various embodiments, the MeHA hydrogel reaches an elasticity of about 2500-5000 Pa after additional exposure to UV radiation, with UV exposure time being proportional to stiffness. In various embodiments, the MeHA hydrogel is a 1% w/v MeHA hydrogel. In various embodiments, the anchorage-dependent cell is a mesenchymal stem cell, a human embryonic stem cell, or a human induced pluripotent stem cell that has been matured to resemble a mammary epithelial cell. In various embodiments, the committed cell type is a mammary epithelial cell.

In another aspect, the invention provides a method of mimicking progression of human heart disease or heart attack in a hydrogel. The method includes providing a MeHA hydrogel having an elasticity defined by elastic constant E (also referred to as “Young's Modulus”), wherein the MeHA hydrogel comprises a photoinitiator such as Irgacure 2959; exposing the MeHA hydrogel to UV radiation for sufficient time to achieve an elasticity of about 10 kiloPascal (kPa); seeding the MeHA hydrogel with an anchorage-dependent cell and allowing the cell to differentiate into a cardiomyocyte or directly using a cardiomyocyte; and thereafter exposing the MeHA hydrogel to additional photoinitiator and additional UV radiation. In various embodiments, the anchorage-dependent cell is a mesenchymal stem cell, a human embryonic stem cell, or a human induced pluripotent stem cell that has been differentiated into a cardiomyocyte. In various embodiments, at day 2 after culturing, hypoxia is induced in the hydrogel. In various embodiments, at day 5 after culturing, the additional UV radiation further crosslinks the MeHA hydrogel to achieve an elasticity of about 50 kPa. In various embodiments, the MeHA hydrogel is a 4% w/v MeHA hydrogel.

In another aspect, the invention provides a method of mimicking progression of human heart disease or heart attack in a hydrogel. The method includes providing a 4% w/v MeHA hydrogel having an elasticity defined by elastic constant E, wherein the MeHA hydrogel comprises a photoinitiator such as Irgacure 2959, exposing the MeHA hydrogel to UV radiation for sufficient time to achieve an elasticity of about 10 kiloPascal (kPa), seeding the MeHA hydrogel with an anchorage-dependent cell, allowing the cell to differentiate into a cardiomyocyte or directly using a cardiomyocyte, culturing the cardiomyocyte, inducing hypoxia in the culture at day 2 after culturing, and exposing the MeHA hydrogel to additional photoinitiator and additional UV radiation at day 5 after culturing to achieve an elasticity of about 50 kPa in the MeHA hydrogel. In various embodiments, the MeHA hydrogel is overlayed with Matrigel after seeding and prior to culturing. In various embodiments, the anchorage-dependent cell is a mesenchymal stem cell, a human embryonic stem cell, or a human induced pluripotent stem cell that has been differentiated into a cardiomyocyte.

In another aspect, the invention provides a system or device for screening compounds for treating breast cancer in a subject. This cell-based assay (i.e., device) includes a solid substrate having disposed thereon a 1% w/v MeHA hydrogel having an elasticity defined by elastic constant E also referred to as a “Young's modulus,” wherein the MeHA hydrogel comprises a photoinitiator and the MeHA hydrogel is exposed to UV radiation for sufficient time to achieve an elasticity of about 100 Pascal (Pa), and an anchorage-dependent cell seeded within the hydrogel. In various embodiments, the anchorage-dependent cell is a mesenchymal stem cell, a human embryonic stem cell, or a human induced pluripotent stem cell that has been matured to resemble a mammary epithelial cell. In various embodiments, the anchorage-dependent cell is allowed to differentiate into a mammary epithelial cell. After approximately 10 days in culture and the formation of mature acinar structures, the MeHA hydrogel can be stiffened to 2500-5000 Pa with additional exposure to the photoinitiator and UV irradiation. During the cells' response, drugs can be added to determine whether the block the adverse changes that accompany the proliferation and dissemination that occur during cancer metastasis.

In another aspect, the invention provides a system or device for screening compounds for treating heart disease or heart attack. This cell-based assay (i.e., device) includes a solid substrate having disposed thereon a 4% w/v MeHA hydrogel having an elasticity defined by elastic constant E, wherein the MeHA hydrogel comprises a photoinitiator such as Irgacure 2959, and the hydrogel is exposed to UV radiationfor sufficient time to achieve an elasticity of about 10 kiloPascal (kPa), and an anchorage-dependent cell seeded within the hydrogel. In various embodiments, the anchorage-dependent cell is a mesenchymal stem cell, a human embryonic stem cell, or a human induced pluripotent stem cell. In various embodiments, the anchorage-dependent cell is allowed to differentiate into a cardiomyocyte. After the formation of a confluent layer of beating cardiomyocytes, hypoxia can be induced and the MeHA hydrogel may be stiffened 50 kPa by exposing the MeHA hydrogel to additional photoinitaor and additional UV radiation. During the cells' response, drugs can be added to determine whether the block cardiomyocyte that accompanies stiffening during a heart attack.

In another aspect, the invention provides a method for screening compounds for treating breast cancer in a subject. The method includes exposing a system or cell-based assay or device to conditions suitable for culturing the anchorage-dependent cell seeded within the MeHA hydrogel for sufficient time to allow formation of an acinar structure, where the system or device includes a solid substrate having disposed thereon a 1% w/v MeHA hydrogel having an elasticity defined by elastic constant E (also referred to as a “Young's modulus”), wherein the MeHA hydrogel comprises a photoinitiator such as Irgacure 2959, and the hydrogel is exposed to UV radiation for sufficient time to achieve an elasticity of about 100 Pascal (Pa), and an anchorage-dependent cell seeded within the hydrogel. The method further includes exposing the device to additional photoinitiator and additional UV radiation such that additional crosslinking allows the MeHA hydrogel to achieve an elasticity that exceeds 1000 Pa, and contacting the cell with a compound of interest. In various embodiments, after 10 days in culture and the formation of mature acinar structures, the MeHA hydrogel may be stiffened to 2500-5000 Pa. Maintenance of the cell's the acinar structure after contact with the compound, is indicative of a compound useful for treating breast cancer. In various embodiments, the anchorage-dependent cell is a mesenchymal stem cell, a human embryonic stem cell, or a human induced pluripotent stem cell. In various embodiments, the anchorage-dependent cell is allowed to differentiate into a mammary epithelial cell.

In yet another aspect, the invention provides a method for screening compounds for heart disease or heart attack in a subject. The method includes exposing a system or cell-based assay or device to conditions suitable for culturing the anchorage-dependent cell seeded within the MeHA hydrogel for sufficient time to allow formation of contractile cardiomyocytes, where the system or device includes a solid substrate having disposed thereon a 4% w/v MeHA hydrogel having an elasticity defined by elastic constant E, wherein the MeHA hydrogel comprises a photoinitiator such as Irgacure 2959, and the hydrogel is exposed to UV radiation for sufficient time to achieve an elasticity of about 8-17 kiloPascal (kPa), and an anchorage-dependent cell seeded within the MeHA hydrogel. The method further includes exposing the device to additional photoinitiator and additional UV radiation such that the MeHA hydrogel achieves an elasticity of at least 50 kPa, and contacting the cell with a compound of interest. Maintenance of the cells' rhythmic contraction after contact with the compound, is indicative of a compound useful for heart disease or heart attack. After the formation of a confluent layer of beating cardiomyocytes, hypoxia can be induced and the material stiffened 50 kPa by exposing the MeHA hydrogel to additional photoinitaor and additional UV radiation. During the cells' response, drugs can be added to determine whether the block cardiomyocyte that accompanies stiffening during a heart attack.

### BRIEF DESCRIPTION OF THE DRAWINGS

FIGS. 1A and 1B are graphical diagrams showing that dystrophic muscle is stiffer than healthy muscle due to fibrosis.

FIGS. 2A and 2B are pictorial and graphical diagrams showing that myocardial infarcts create spatially dependent changes in matrix.

FIGS. 3A and 3B show that cancer stiffens mammary tissue. Below 500 Pascal (Pa), cells look relatively normal and are in hollow spheres called “acini.” Above 675 Pa, they fill the lumen of these spheres and grow into large sheets of cells resembling a tumor.

FIGS. 4A-4F are pictorial and graphical diagrams showing the chemistry involved (FIG. 4A) and a general polymerization scheme (FIG. 4B) for the MeHA hydrogel. FIG. 4C shows a scheme for using the MeHA hydrogel with mammary epithelial cells and an overlay of a material called Matrigel. Cell responses are shown in FIG. 4D. FIGS. 4E and 4F show what the material looks like and its corresponding stiffness.

FIGS. 5A-5C are pictorial and graphical diagrams showing that UV exposure makes the hydrogels stiffer, thereby mimicking different stages of cancer.

FIGS. 6A-6C are pictorial and graphical diagrams showing that UV exposure makes the hydrogels stiffer, thereby mimicking different stages of cancer.

FIG. 7 is a graphical diagram showing gel stiffness characterization for mimicking heart tissue.

FIG. 8 is a pictorial and graphical diagram showing the protocol for growing cardiomyocytes on the MeHA gel in culture.

FIGS. 9A and 9B are graphical diagrams showing asynchronous contractions of cardiomyocytes after dynamic stiffening. FIG. 9A shows Ca²⁺ waveforms for cardiomyocytes cultured on 11 kPa gels and stiffened gels. FIG. 9B shows the correlation coefficient, which is a measure of beating synchronicity, was significantly higher for cardiomyocytes lacking risk alleles in the 9p21 gene locus (N/N) when cultured on stiffened gels compared to the counterpart cells that have the risk alleles (R/R). Groups with different letters are significantly different from others.

FIG. 10 is a series of pictorial diagrams showing immunofluorescent staining images for connexin 43 for the indicated iPSC-CM patient types and bioreactor conditions. Thus, gap junction remodeling in response to stiffening contributes to asynchronous contractions. Arrowheads indicate regions of functional connexin 43 expression between cardiomyocytes.

FIGS. 11A-11C are a series of graphical diagrams showing that R/R dysfunction also manifested in other Ca²⁺ handling metrics. FIG. 11A shows that normalized peak area, defined as the area under the Ca²⁺ waveform divided by the number of contractions for cardiomyocytes cultured on dynamically stiffened gels compared to 11 kPa gels, was significantly increased in R/R coronary artery disease (CAD) positive cardiomyocytes. FIG. 11B shows that normalized peak amplitude, defined as the height of the largest contraction, was reduced for R/R cardiomyocytes compared to N/N cardiomyocytes. FIG. 11C shows that normalized frequency, defined as the number of contractions, was increased in R/R cardiomyocytes compared to N/N cardiomyocytes. Groups with different letters are significantly different from others.

FIGS. 12A-12C are pictorial and graphical diagrams showing that loss of sarcomeric organization in R/R due to gel stiffening contribute to decreased peak area. FIG. 12A shows immunofluorescent staining images for α-actinin, a sarcomere protein in the contractile apparatus of cardiomyocytes. Arrowheads indicate functional sarcomeres. FIG. 12B shows that the percentage of cardiomyocytes with organized α-actinin pattern in greater than one-fourth of total cell area. FIG. 12C shows the quantification of sarcomere spacing.

### DETAILED DESCRIPTION OF THE INVENTION

The present invention is based on the finding that the properties of certain hydrogels may be altered to mimic progressive environmental changes of diseased tissue.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

The term “comprising,” which is used interchangeably with “including,” “containing,” or “characterized by,” is inclusive or open-ended language and does not exclude additional, unrecited elements or method steps. The phrase “consisting of” excludes any element, step, or ingredient not specified in the claim. The phrase “consisting essentially of” limits the scope of a claim to the specified materials or steps and those that do not materially affect the basic and novel characteristics of the claimed invention. The present disclosure contemplates embodiments of the invention compositions and methods corresponding to the scope of each of these phrases. Thus, a composition or method comprising recited elements or steps contemplates particular embodiments in which the composition or method consists essentially of or consists of those elements or steps.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

The term “cancer” as used herein, includes any cell having uncontrolled and/or abnormal rate of division that then invade and destroy the surrounding tissues. Cancer is a multistep process that can be defined in terms of stages of malignancy wherein the normal orderly progression is aberrant. In broad stages, normal tissue may begin to show signs of hyperplasia or show signs of neoplasia. As used herein, “hyperplasia” refers to cells that exhibit abnormal multiplication or abnormal arrangement in a tissue. Included in the term hyperplasia, are benign cellular proliferative disorders, including benign tumors. As used herein, “proliferating” and “proliferation” refer to cells undergoing mitosis. As used herein “neoplasia” refers to abnormal new growth, which results in a tumor. Unlike hyperplasia, neoplastic proliferation persists even in the absence of the original stimulus and characterized as uncontrolled and progressive. Malignant neoplasms, or malignant tumors, are distinguished from benign tumors in that the former show a greater degree of anaplasia and have the properties of invasion and metastasis. As used herein, “metastasis” refers to the distant spread of a malignant tumor from its sight of origin. Cancer cells may metastasize through the bloodstream, through the lymphatic system, across body cavities, or any combination thereof. Exemplary cancers include, but are not limited to, neuroblastoma and breast cancer.

Standard techniques for growing cells, separating cells, analyzing gene expression, determining cell surface biomarkers and where relevant, cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described by Freshney, R. I., Culture of Animal Cells: A Manual of Basic Technique, 5e. 2007, John Wiley & Sons, Inc., New Jersey Sambrook et al., 1989 Molecular Cloning, Second Edition, Cold Spring Harbor Laboratory, Plainview, N.Y.; Maniatis et al., 1982 Molecular Cloning, Cold Spring Harbor Laboratory, Plainview, N.Y.; Wu (Ed.) 1993 Meth. Enzymol. 218, Part I; Wu (Ed.) 1979 Meth. Enzymol. 68; Wu et al., (Eds.) 1983 Meth. Enzymol. 100 and 101; Grossman and Moldave (Eds.) 1980 Meth. Enzymol. 65; Miller (ed.) 1972 Experiments in Molecular Genetics, Cold Spring Harbor Laboratory, Cold Spring Harbor, N.Y.; Old and Primrose, 1981 Principles of Gene Manipulation, University of California Press, Berkeley; Schleif and Wensink, 1982 Practical Methods in Molecular Biology; Glover (Ed.) 1985 DNA Cloning Vol. I and II, IRL Press, Oxford, UK; Hames and Higgins (Eds.) 1985 Nucleic Acid Hybridization, IRL Press, Oxford, UK; and Setlow and Hollaender 1979 Genetic Engineering: Principles and Methods, Vols. 1-4, Plenum Press, New York. Abbreviations and nomenclature, where employed, are deemed standard in the field and commonly used in professional journals such as those cited herein.

The term “human Pluripotent Stem Cells” or “hPSCs,” of which “human Embryonic Stem Cells” or “hESCs” and “human induced pluripotent stem cells” or “hiPSCs” are a subset, refers to cells derived from pre-embryonic, embryonic, or fetal tissue at any time after fertilization, and have the characteristic of being capable under appropriate conditions of producing progeny of several different cell types that are derivatives of all of the three germinal layers (endoderm, mesoderm and ectoderm). The term includes both established lines of stem cells of various kinds, and cells obtained from primary tissue that are pluripotent in the manner described. Included in the definition of pluripotent stem cells (PSCs) are embryonic cells of various types, especially including human embryonic stem cells (hESCs), described by Thomson et al. (Science 282: 1145, 1998). Other types of pluripotent cells are also included in the term. The term “Human Pluripotent Stem Cells” includes stem cells which may be obtained from human umbilical cord or placental blood as well as human placental tissue. Any cells of primate origin that are capable of producing progeny that are derivatives of all three germinal layers are included, regardless of whether they were derived from embryonic tissue, fetal, or other sources.

An “induced pluripotent stem cell” refers to a pluripotent stem cell artificially (e.g., non-naturally, in a laboratory setting) derived from a non-pluripotent cell. A “non-pluripotent cell” can be a cell of lesser potency to self-renew and differentiate than a pluripotent stem cell. Cells of lesser potency can be, but are not limited to adult stem cells, tissue specific progenitor cells, primary or secondary cells. An adult stem cell is an undifferentiated cell found throughout the body after embryonic development.

A used herein, the term “mesenchymal stem cell” or “MSC” refers to a multipotent stromal cell that can differentiate into a variety of cell types including: osteoblasts (bone cells), chondrocytes (cartilage cells), myocytes (muscle cells), and adipocytes (fat cells).

The term “differentiation” is used to describe a process wherein an unspecialized (“uncommitted”) or less specialized cell acquires the features of a more specialized cell such as, for example, human embryonic stem cell derived epithelial cell (hESC-EC), human embryonic stem cell derived mesenchymal cell (hESC-MC), or where a more specialized intermediate cell, such as a mesenchymal cell (hES-MC) or epithelial cell (hES-EC) becomes an even more specialized cell such as a bone cell, a cartilage cell or a smooth muscle cell. A differentiated or differentiation-induced cell is one that has taken on a more specialized (“committed”) position within the lineage of a cell. The term “committed,” when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type. As used herein, the lineage of a cell defines the heredity of the cell, i.e., which cells it came from and what cells it can give rise to. The lineage of a cell places the cell within a hereditary scheme of development and differentiation. A lineage-specific marker refers to a characteristic specifically associated with the phenotype of cells of a lineage of interest and can be used to assess the differentiation of an uncommitted cell to the lineage of interest.

As used herein when referring to a cell, cell line, cell culture or population of cells, the term “isolated” refers to being substantially separated from the natural source of the cells such that the cell, cell line, cell culture, or population of cells are capable of being cultured in vitro. In addition, the term “isolating” may be used to refer to the physical selection of one or more cells out of a group of two or more cells, wherein the cells are selected based on cell morphology and/or the expression of various markers. It is noted herein that in various aspects of the present invention, one of the principal benefits is that isolation of cells, because of the levels of confluence and population consistency, do not require a separate isolation technique or step. Within this context, the term “isolating” may simply refer to the passaging of cells without further isolation steps being used to provide unexpected consistency of the final isolated cell population.

The term “hydrogel” as used herein refers to three-dimensional hydrophilic polymeric networks. Hydrogels have high water content, providing an environment sufficient transportation of nutrients and waste products, which is essential for cell growth. Thus, a hydrogel is a 3-dimensional network of natural or synthetic hydrophilic polymer chains in which water (up to 99%) is the dispersion medium. The high water content of the hydrogels render the material biocompatible and provide a flexibility comparable to that of living tissue. Hydrogels are thus of interest in biomedical engineering and have been prepared by physical or chemical crosslinking of hydrophilic natural or synthetic polymers.

As used herein, the terms “acinus”, “acini”, or acinar structure” are used to describe a cluster of spherical monolayers of epithelial cells that enclose a central lumen.

In vivo, the extracellular matrix (ECM) provides both mechanical support for surrounding cells and a variety of biochemical and biophysical signals that influence cellular behavior. These are largely the result of the ECM composition that includes adhesive glycoproteins, fibrous matrix proteins, proteoglycans, and glycosaminoglycans (Badylak, 2005). These signals are coupled in the body and together they create a 3-dimensional microenvironment for cell growth (Cukierman et al., 2001).

Regardless of geometry, the intrinsic resistance of a solid to a stress is measured by the solid's elastic (or Young's) modulus E, which is most simply obtained by applying a force—such as hanging a weight—to a section of tissue or other material and then measuring the relative change in length or strain. Another common method to obtain E involves controlled macro- or micro-indentation, including atomic force microscopy (AFM). The elastic modulus E is discussed, e.g., by Rotsch, et al., “Dimensional and mechanical dynamics of active and stable edges in motile fibroblasts investigated by using atomic force microscopy.” Proc. Natl. Acad. Sci. USA, 1999. 96(3): p. 921-926; Radmacher, M., “Measuring the elastic properties of living cells by the atomic force microscope.” Methods Cell Biol, 2002. 68: p. 67-90; Engler et al., 2004c, supra. Many tissues and biomaterials exhibit a relatively linear stress versus strain relation up to small strains of about 10 to 20%. The slope E of stress versus strain is relatively constant at the small strains exerted by cells (Lo et al., 2000, supra), although stiffening (increased E) at higher strains is the norm (Storm et al., Nature 435:191 (2005); Fung, A First Course in Continuum Mechanics: For Physical and Biological Engineers and Scientists (Prentice Hall, Englewood Cliffs, N.J., ed. 3, 1994).

Nonetheless, microscopic views of both natural and synthetic matrices (e.g., collagen fibrils and polymer-based mimetics (Stevens et al., Science 310:1135 (2005)), suggest that there are many subtleties to tissue mechanics, particularly concerning the length and time scales of greatest relevance to cell sensing. The elastic resistance that a cell ‘feels’ when it attaches to a substrate is governed by the elastic constant E of the substrate or tissue microenvironment. Sample preparation is also critical; for example, macroscopic elastic moduli measurements of whole brain can vary 2-fold or more, depending on sample preparation, perfusion, etc. (Gefen et al., J. Biomech. 37:1339 (2004)). In addition, many single or multi-cell probing methods involve high-frequency stressing (Hu et al., Am. J. Physiol. Cell Physiol. 287:C1184 (2004)), whereas relevant time scales for cell-exerted strains seem likely to range from seconds to hours, motivating long time studies of cell rheology (Bao et al., Nat. Mater. 2:715 (2003); Wottawah etal., Phys. Rev. Lett. 94:098103 (2005)).

Correlations have long been made between increased cell adhesion and increased cell contractility (e.g., Leader et al., J. Cell Sci. 64:1 (1983)), but it now seems clear that tactile sensing of substrate stiffness feeds back on adhesion and cytoskeleton, as well as on net contractile forces, for many cell types. Seminal studies on epithelial cells and fibroblasts exploited inert polyacrylamide gels with a thin coating of covalently attached collagen (Pelham et al., 1997, supra). This adhesive ligand allows the cells to attach and, by controlling the extent of polymer cross-linking in the gels, E can be adjusted over several orders of magnitude, from extremely soft to stiff

The present invention is therefore based on the observation that cell behavior on compliant hydrogels often more closely approximates in vivo behavior compared to cells on rigid culture substrates, e.g., glass or plastic. This occurs in part because cells can ‘feel’ the hydrogel's elastic modulus. The modulus of elasticity E, or stiffness, is a characteristic of the ECM that certain anchorage-dependent cells can sense and respond to with a variety of cellular processes (Discher et al., 2005). Thus, E of a material represents the intrinsic resistance of organs and tissues to stress, and in its simplest mathematical form can be expressed as the tensile stress, or force applied per unit area, divided by the resultant strain, or relative change in length (Discher et al., 2005). Though highly nonlinear (Fung, 1993), at the physiologically appropriate strains, the degree of stiffness varies dramatically between tissues: brain (E brain 0.1-1 kPa) is clearly softer than striated skeletal muscle (E muscle 8-17 kPa), which is less stiff than precalcified bone (E precalcified bone=25-40 kPa; Engler et al., 2006). At the cellular level, such changes in substrate elasticity have been observed to influence several cellular behaviors, including cell proliferation, locomotion, adhesion, spreading, morphology, striation, and even differentiation of stem cells (Pelham and Wang, 1997; Wang et al., 2000; Flanagan et al., 2002; Engler et al., 2004b, 2006; Khatiwala et al., 2006; Reinhart-King et al., 2005; Discher et al., 2009). That said, these properties are not static within the body; they are often displayed in highly complex gradients, such as those of stiffness at tissue interfaces.

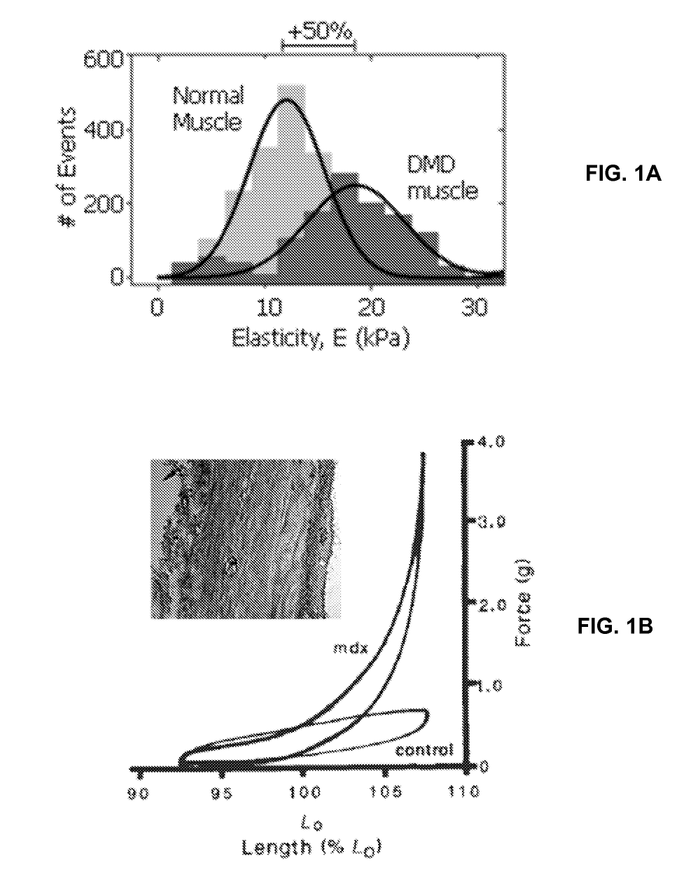

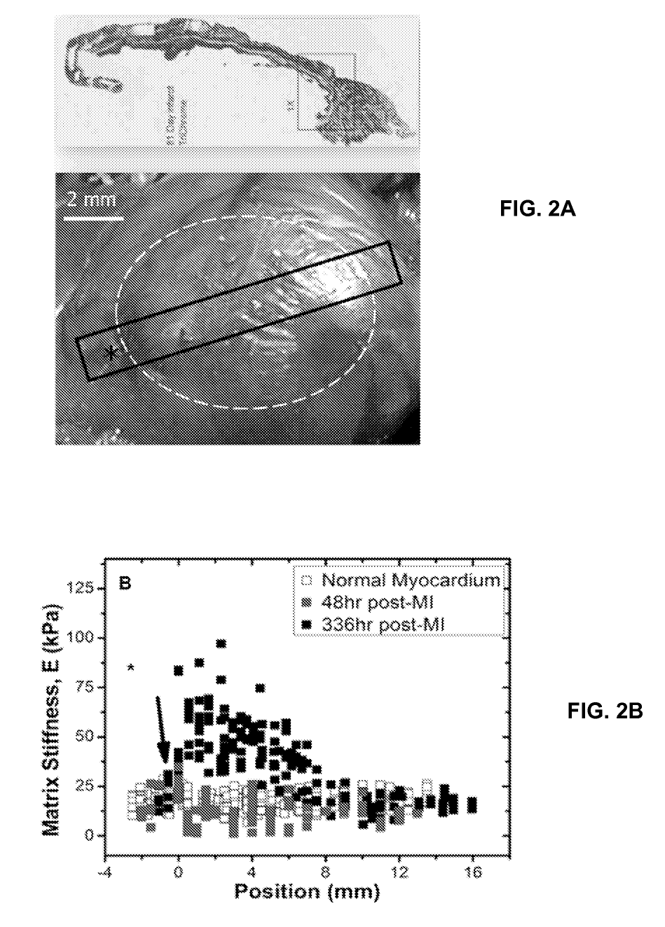

Many fibrotic diseases cause the relevant tissue to stiffen. As shown in FIG. 1, muscle tissue from patients with Duchenne Muscular Dystrophy (DMD) have been observed to become stiffer, as shown by atomic force microscopy (left) and bulk tensile loading (right). This is also shown for rats that have had “artificially” been given a heart attack (FIG. 2).

Mature, contractile heart cells, i.e., cardiomyocytes, have traditionally been cultured on thick collagen gels to maintain rhythmic contraction (Bird, et al., 2003; Sanger, et al., 2005), but more recently it was shown with synthetic gels that stiffness is a critical regulator of contraction. This may be due in part to its modulation of cytoskeletal assembly in the form of myofibril striation and alignment, both of which can affect beating rate. As with most other cell types, these behaviors in culture become most in vivo-like when cells are grown on a substrate which mimics the stiffness of their native environment. For example on a 10 kPa hydrogel, which mimics adult myocardial stiffness and is similar to other muscle types, intra- and extra-cellular strains become matched and can prolong rhythmic beating in culture. When too stiff or soft, cardiomyocytes overstrain themselves or do little work on the substrate, respectively, which in both cases result in few striated myofibrils and a loss of rhythmic contraction. Thus, these are cardiovascular applications where providing a hydrogel that stiffens over time can be useful to mimic the changes in stiffness of these tissues.

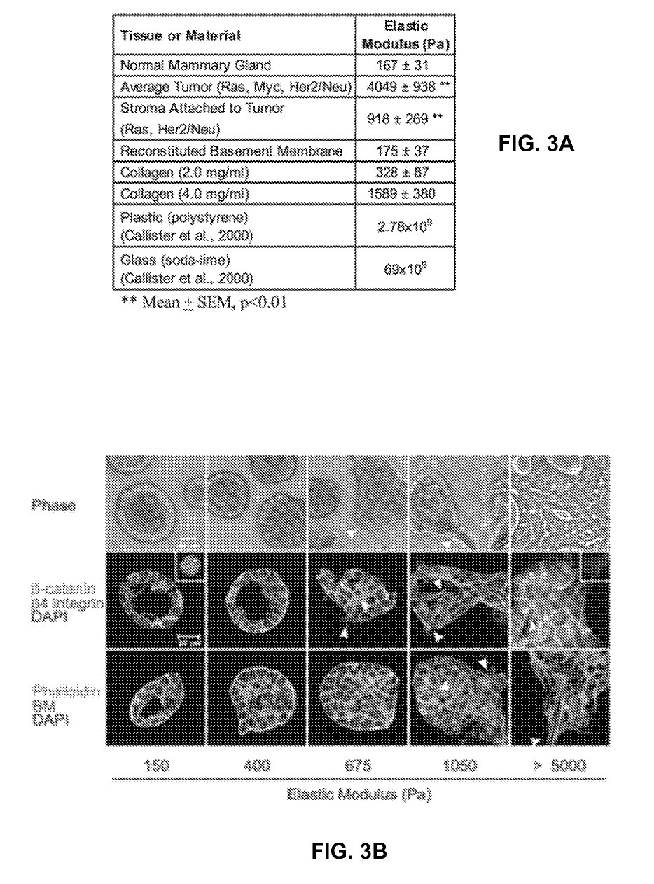

As another example, mammary tissue is known to stiffen with the onset of cancer (i.e., a subject can feel a lump in mammary tissue where a tumor exists) given all of the changes that occur (e.g., more extracellular matrix is secreted). As shown in FIG. 3, normal stiffness of mammary tissue is ˜150 Pa, whereas at above 675 Pa, the “normal” mammary tissue becomes cancerous from the ‘stiff signals’ they receive from their environment, resulting in the formation of a tumor that stiffens to ˜5700 Pa.

Thus, the fabrication of matrix substrates with a defined modulus of elasticity can be a useful technique to study the interactions of cells with their biophysical microenvironment. Matrix substrates composed of polyacrylamide hydrogels have an easily quantifiable elasticity that can be changed by adjusting the relative concentrations of its monomer, acrylamide, and cross-linker, bis-acrylamide.

To minimize variability, it is beneficial if the materials and methods for making the gels are reproducible (see, e.g., US Pub. Nos. 2007/0190646 and 2010/0015709), and perhaps, produced by an automated means to reduce introduced variability. Gel monomers are mixed with agents that induce polymerization and then are poured into a mold that dictates the size and shape of the polymerized gel. For example, the catalyzed liquid gel monomer can be poured between glass plates separated uniformly over the entire surfaces thereof to produce a square or rectangular slab gel. The glass plates, separated by about a millimeter or a fraction thereof, are held in place until the gel is formed. The concentrations of polyacrylamide gels used in electrophoresis are generally stated in terms of % T (the total percentage of acrylamide in the gel by weight) and % C (the proportion of the total acrylamide that is accounted for by the cross-linker used). In various embodiments, N,N′-methylenebisacrylamide (“bis”) may be used as a cross-linker.

Accordingly, the present invention provides methods of mimicking environmental changes of tissues through use of a hydrogel for culturing cells. In one embodiment, a polyacrylamide (PA) hydrogel that has static mechanical properties with time is provided. In another embodiment, two types of hyaluronic acid (HA) hydrogels that have dynamic properties with time are provided.

The first HA hydrogel uses polyethylene glycol-diacrylate and HS that has 35% thiol modification to achieve time-dependent crosslinking via a michael-type addition reaction. The second HA hydrogel uses UV polymerization, a photoinitiator, and methacrylate modified HA. (Guvendiren and Burdick, “Stiffening hydrogels to probe short- and long-term cellular responses to dynamic mechanics,” Nature Communications, 2012, incorporated herein by reference). While the first HA hydrogel permits gradual stiffening, the second HA hydrogel using UV-activated, methacrylate-based crosslinking that allows for crosslinking to be “on demand,” meaning that multiple crosslinking steps can be performed at the user's choosing vs. continuous crosslinking with thiolated. In various embodiments, the photoinitiator is a phot-activated free-radical donor. An exemplary photoinitiator useful in the present invention is Irgacure 2959.

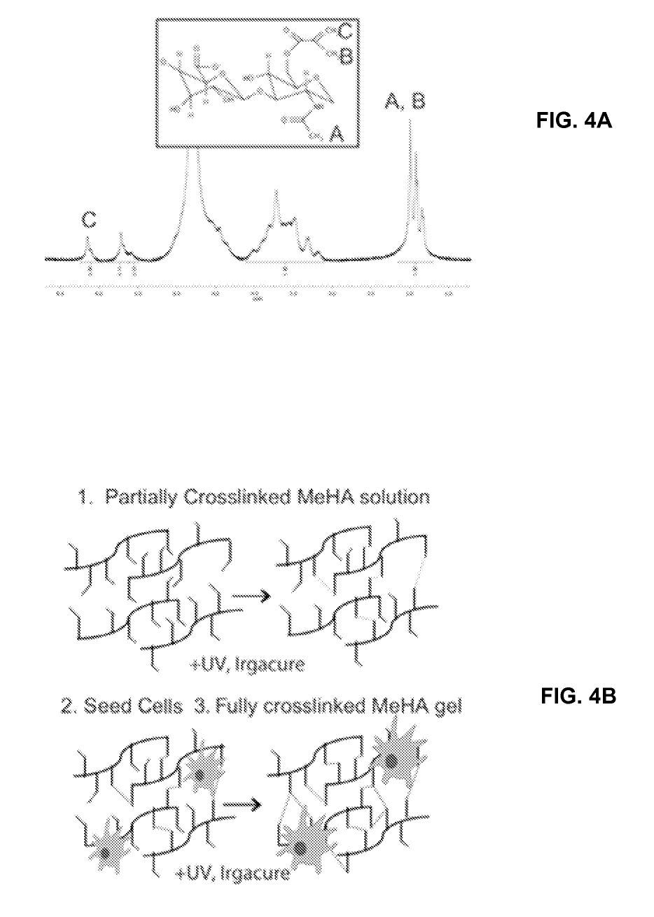

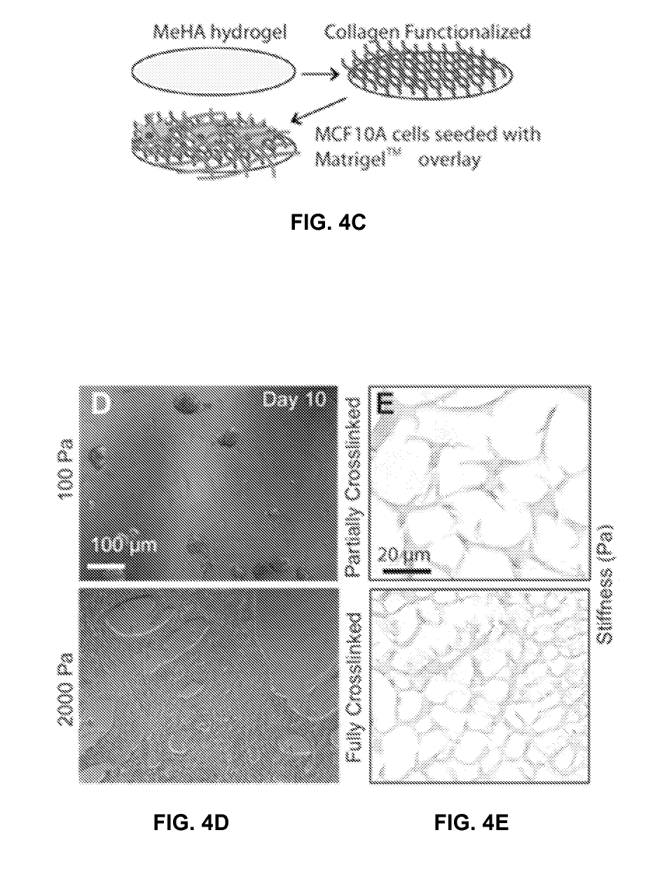

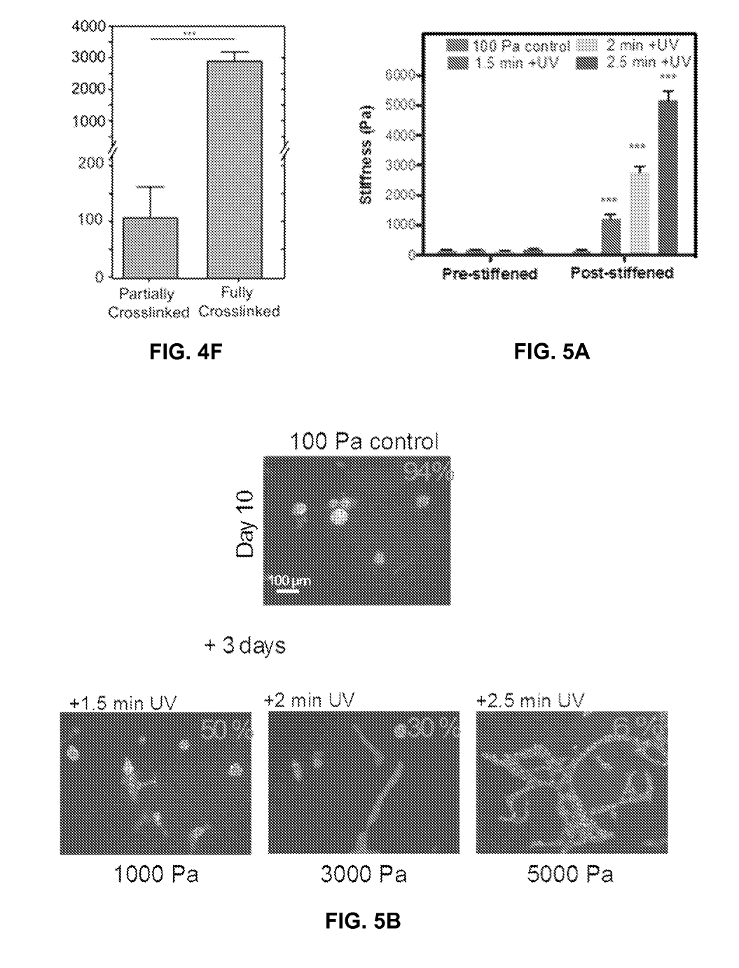

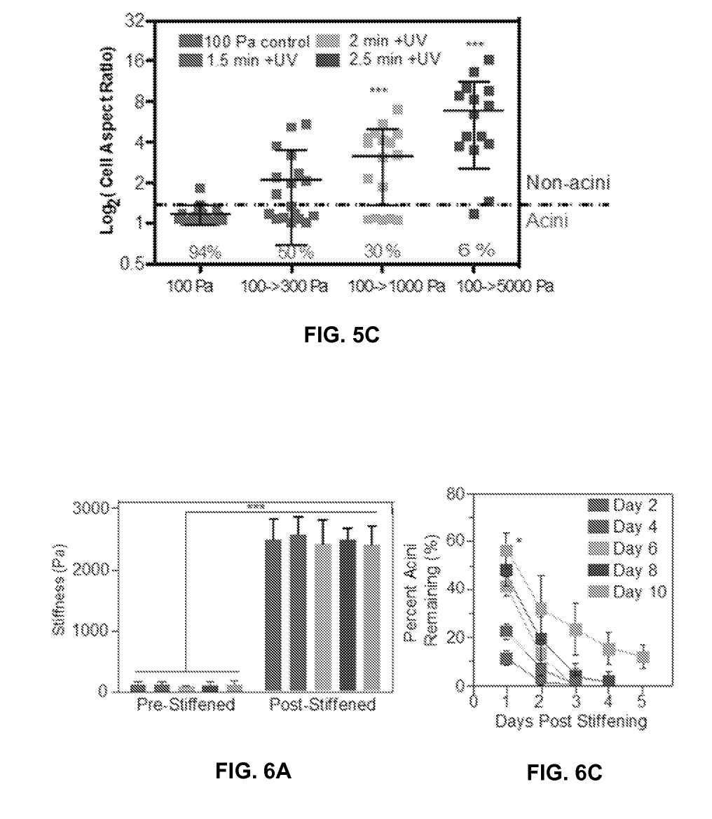

Accordingly, in various embodiments, the present invention provides use of a 1% w/v MeHA hydrogel with 1 minute UV polymerization to achieve a hydrogel that has an elasticity of about 100 Pa. FIG. 4A shows the chemistry used, while FIG. 4B shows a general polymerization scheme for the MeHA hydrogel. FIG. 4C shows a scheme for using the MeHA hydrogel with mammary epithelial cells and an overlay of a material called Matrigel.

FIGS. 4E and 4F show what the material looks like and its corresponding stiffness. Cell responses are shown in FIG. 4D.

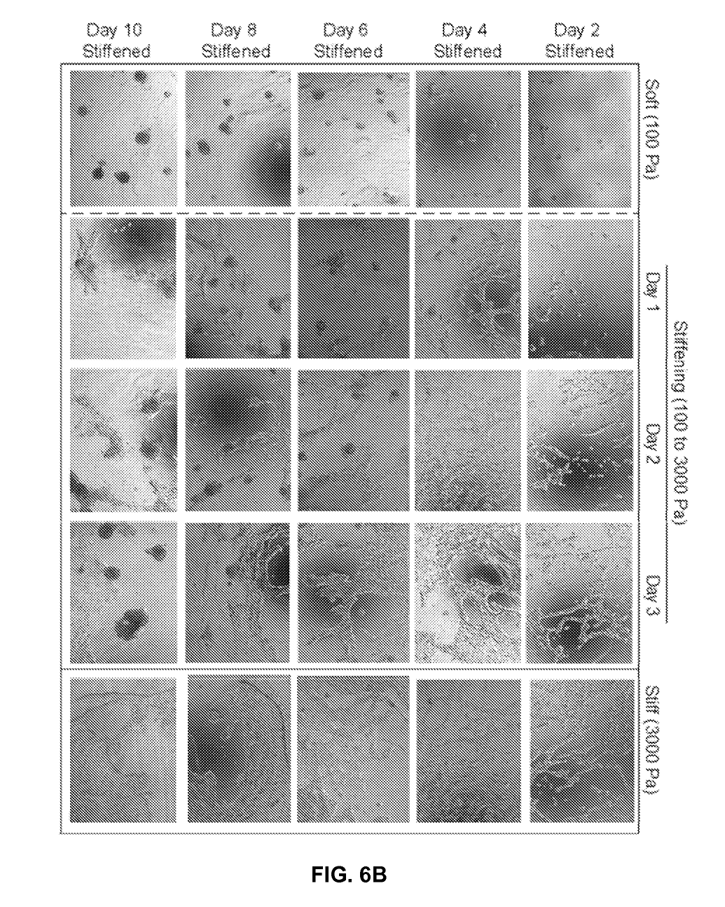

FIG. 6A shows that this is a tunable system, meaning that additional UV exposure makes the hydrogels stiffer, thereby mimicking different stages of cancer progression. Quantification of cell responses is shown in FIG. 6C. By dynamically stiffening the system vs. having it be stiff and static the entire time, the present invention demonstrates that the cells are less receptive to stiffness-induced tumor transformation than was previously thought. The same is true if the stiffness is adjusted to the same degree (e.g., 100 to 2500 Pa; see FIG. 6A), but do that with mammary epithelial cells that are of varying maturity (mature hollow spheres in adult mammary tissue are called “acini”) as shown in FIG. 6B. Once the cells form these mature structures by day 10, 10-25% of the structures resist stiffening and remain hollow spheres as shown in FIG. 6C.

Thus, the MeHA hydrogel system provided herein mirrors the transitions from soft mammary tissue to stiffer tumor tissue and does a better job of mirroring what occurs in vivo in women that develop mammary tumors. Prior disease models do not recapitulate this, and so their data does not correctly estimate the amount of stiffening required to get tumor formation from healthy cells.

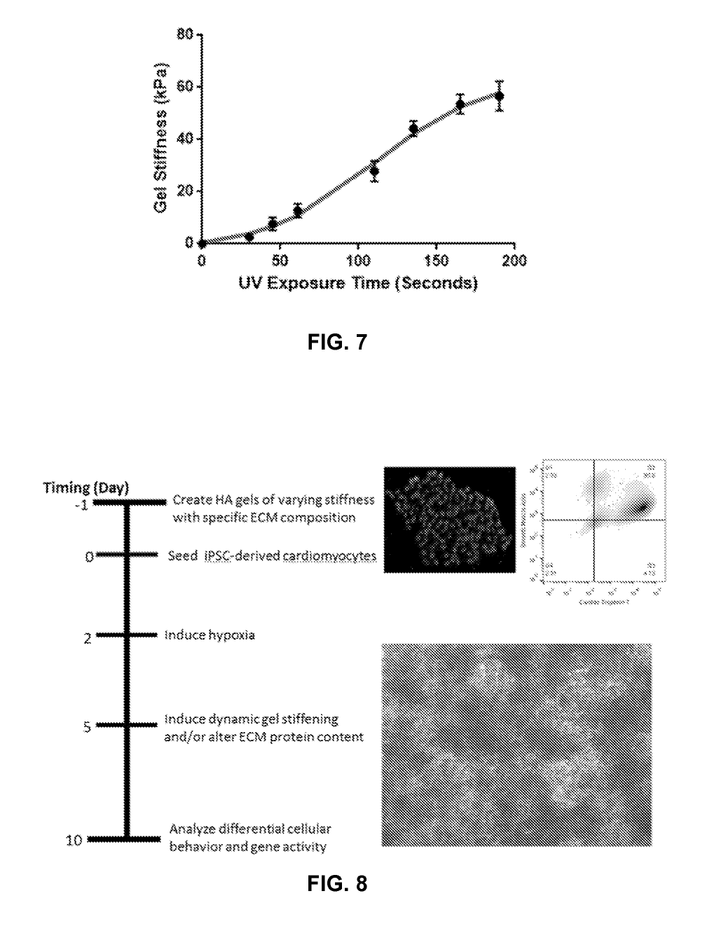

For modeling the transitions from healthy heart stiffness (i.e., ˜10 kPa, as defined in FIG. 2) to diseased stiffness (i.e., ˜50 kPa from FIG. 2), the present invention provides use of a 4% w/v MeHA hydrogel with a photoinitiator such as Irgacure 2595, with the amount of UV exposure shown in FIG. 7.

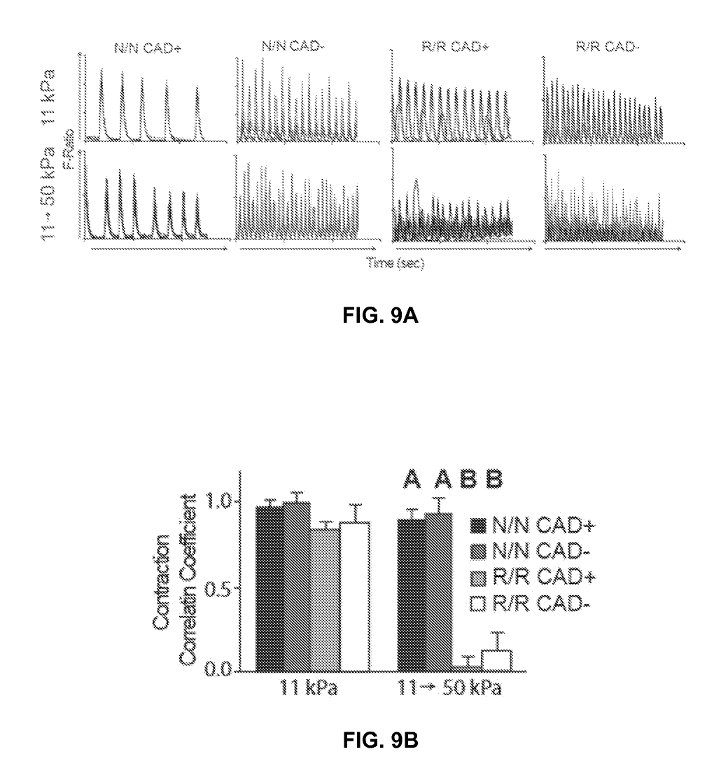

Using the protocol outlined in FIG. 8, stem cells (images at top) are seeded and differentiated into cardiomyocytes (as defined in the plot—cardiomyocytes are in quadrant 2), and then grown on the MeHA gel in culture. At day 2, hypoxia is induced, and at day 5, the gel is stiffened from 10 to 50 kPa. As shown in FIGS. 9A-9C, calcium dyes, indicative of cellular contraction, demonstrate that cardiomyocytes that have single nucleotide polymorphisms (SNPs) in the 9p21 gene locus making them homozygous risk/risk (R/R) for CAD as well as myocytes which are non-risk/non-risk (N/N) beat rhythmically. However, the same cells on the stiffened hydrogel do not. Accordingly, the HA hydrogel described herein, which transitions from normal stiffness, where the cells can acclimate to their environment first, to a heart attack stiffness, better mirrors what occurs in vivo than static culture systems like PA hydrogels or tissue culture plastic, where 95% of all cell culture is done.

Thus, some of the factors identified and studied to be important to achieve the physical properties to direct the desired tissue status on a chip include (but not limited to): choice of the hydrogels (such as polyacrylamide and hyaluronic acid based) and cross-linkers (such as polyethylene glycol-diacrylate or methacrylate-based), their concentrations (singly or mixed), numbers and types of modifications, mechanisms and control of crosslinking reactions, duration of crosslinking, and others.

Accordingly, in another aspect, the invention provides a system or device for screening compounds for treating breast cancer in a subject. The device includes a solid substrate having disposed thereon a 1% w/v MeHA hydrogel having an elasticity defined by elastic constant E also referred to as a “Young's modulus,” wherein the MeHA hydrogel is mixed with a photoinitiator and is exposed to UV radiation for sufficient time to achieve an elasticity of about 100 Pascal (Pa), and an anchorage-dependent cell seeded within the hydrogel. In various embodiments, the anchorage-dependent cell is a mesenchymal stem cell, a human embryonic stem cell, or a human induced pluripotent stem cell that has been matured to resemble a mammary epithelial cell. In various embodiments, the anchorage-dependent cell is allowed to differentiate into a mammary epithelial cell. After approximately 10 days in culture and the formation of mature acinar structures, the MeHA hydrogel can be stiffened to 2500-5000 Pa by additional exposure to the photoinitiator, which allows the photoinitiator to absorb into the hydrogel, followed by additional exposure to UV radiation. During the cells' response, drugs can be added to determine whether the block the adverse changes that accompany the proliferation and dissemination that occur during cancer metastasis.

Similarly, a system or device for screening compounds for treating heart disease or heart attack is also provided. In this embodiment, the device includes a solid substrate having disposed thereon a 4% w/v MeHA hydrogel having an elasticity defined by elastic constant E, wherein the MeHA hydrogel is mixed with a photoinitiator and is exposed to UV radiation for sufficient time to achieve an elasticity of about 10 kiloPascal (kPa), and an anchorage-dependent cell seeded within the hydrogel. In various embodiments, the anchorage-dependent cell is a mesenchymal stem cell, a human embryonic stem cell, or a human induced pluripotent stem cell. In various embodiments, the anchorage-dependent cell is allowed to differentiate into a cardiomyocyte. After the formation of a confluent layer of beating cardiomyocytes, hypoxia can be induced and the material stiffened 50 kPa by additional exposure to the photoinitiator, which allows the photoinitiator to absorb into the hydrogel, followed by additional exposure to UV radiation. During the cells' response, drugs can be added to determine whether the block cardiomyocyte that accompanies stiffening during a heart attack.

The methods, systems, and devices of the invention are adaptable to a wide variety of assays, such as screening assays for compounds or agents useful in treating diseases. Accordingly, the invention provides a method for screening compounds for treating breast cancer in a subject. The method includes exposing a system or device to conditions suitable for culturing the anchorage-dependent cell seeded within the MeHA hydrogel for sufficient time to allow formation of an acinar structure, where the system or device includes a solid substrate having disposed thereon a 1% w/v MeHA hydrogel having an elasticity defined by elastic constant E also referred to as a “Young's modulus,” wherein the MeHA hydrogel contains a photoinitiator such as Irgacure 2959, and is exposed to UV radiation for sufficient time to achieve an elasticity of about 100 Pascal (Pa), and an anchorage-dependent cell that is seeded within the hydrogel. The method further includes exposing the device to additional photoinitiator and additional UV radiation such that the MeHA hydrogel achieves an elasticity that exceeds 1000 Pa, and contacting the cell with a compound of interest. In various embodiments, after 10 days in culture and the formation of mature acinar structures, the MeHA hydrogel may be stiffened to 2500-5000 Pa. Maintenance of the cell's the acinar structure after contact with the compound, is indicative of a compound useful for treating breast cancer.

The methods described herein can be modified for high throughput use and for better disease modeling in vitro, thus allowing the examination of a plurality (i.e., 2, 3, 4, or more) of compounds or test agents, which independently can be the same or different, in parallel. As such, a high throughput format allows for the examination of two, three, four, etc., different compounds or test agents, alone or in combination, on the cells such that the best (most effective) agent or combination of agents can be identified for development into a therapeutic drug. Further, a high throughput format allows, for example, control samples (positive controls and or negative controls) to be run in parallel with test samples.

The methods provided herein may be used to provide a tissue on a chip for (1) drug discovery including pre-clinical studies; (2) disease modeling for research; and/or (3) individualized medicine. Accordingly, in another aspect, the invention provides a device for screening compounds for treating breast cancer in a subject. The device includes a solid substrate having disposed thereon a 1% w/v MeHA hydrogel, wherein the MeHA hydrogel contains a photoinitiator such as Irgacure 2959, and is exposed to UV radiation for about 1 minute to achieve an elasticity of about 100 Pascal (Pa). Likewise, the invention provides a device for screening compounds for treating heart disease or heart attack. The device includes a solid substrate having disposed thereon a 4% w/v MeHA hydrogel, wherein the MeHA hydrogel contains a photoinitiator such as Irgacure 2959, and is exposed to UV radiation for about 1 minute to achieve a hardness of about 10 kiloPascal (kPa).

The following examples are intended to illustrate but not limit the invention.

### EXAMPLE 1

### Polyacrylamide (PA) Hydrogel as a Static Substrate

PA gels are produced in this protocol by mixing various acrylamide and bis-acrylamide concentrations and inducing free radical polymerization. PA gel modulus of elasticity was quantified using atomic force microscopy (AFM), which is a nano-indentation method of calculating elasticity. This technique has been extensively detailed elsewhere (Rotsch et al., 1999; Rotsch and Radmacher, 2000).

Details of the PA hydrogel fabrication can be found in Tse and Engler, “Preparation of Hydrogel Substrates with Tunable Mechanical Properties,” Current Protocols in Cell Biology (2010), incorporated herein by reference.

Materials used are: 0.1 M NaOH; Distilled H₂O; 3-Aminopropyltriethoxysilane (APES); 0.5% (v/v) glutaraldehyde in phosphate-buffered saline (PBS; Cellgro, cat. no. 46-013-CM); Dichlorodimethylsilane (DCDMS); 40% (w/v) acrylamide stock solution (Sigma-Aldrich, cat. no. A4058); 2% (w/v) bis-acrylamide stock solution (Sigma-Aldrich, cat. no. M1533); Phosphate-buffered saline (PBS); optional Tetramethylethylenediamine (TEMED); 10% (wv) ammonium persulfate (APS); 25-mm circular coverslips (for 6-well plate); Hot plate 35-mm petri dish(es); Kimwipes; 75 mm glass slides; Vacuum desiccator; Vortex mixer; 6-well plate.

The hydrogel was prepared as follows: Place 25-mm coverslip(s) on a hot plate and add 500 μl of 0.1 M NaOH to the coverslip so that the solution covers the entire glass surface. Heat the coverslip with solution at 80° C. until the liquid is evaporated. The solution should not boil, and there should be a thin semi-transparent film of NaOH remaining on the coverslip(s) after evaporation. Repeat step 1 by diluting the NaOH by adding 500 μl of distilled H₂O to the coverslip and heating the solution at 80° C. until the film of NaOH is uniform. This step should be performed if and only if steps 1 and 2 resulted in a non-uniform film. A uniform film of NaOH is important for uniform gel attachment. Place coverslip(s) in a nitrogen-filled tent. Add 200 μl of APES to the surface of the coverslip(s). Allow 5 min for the APES to react. If a nitrogen tent is unavailable, this step can be done in the fume hood. Since APES will react with the oxygen in the air, use 250 μl of APES to compensate for the loss of reactivity. A thin film will likely result on the surface of the APES solution and additional washing cycles in step 6 may be necessary to remove it. Rinse the coverslip(s) with distilled H₂O under the distilled H₂O tap to ensure both the top and bottom of the coverslip(s) is rinsed. It is important to completely rinse off the unreacted APES, for it will create an orange-brown precipitate with glutaraldehyde (see step 8) that fluoresces under UV light and can thus interfere with immunostaining techniques. Place the coverslip(s) in distilled H₂O into a petri dish and rinse the coverslips twice, each time in 10 ml (or enough to immerse the coverslip) distilled water for 5 min each. Aspirate the second distilled H₂O wash solution and add 10 ml (or enough to immerse the coverslip) of 0.5% glutaraldehyde in PBS. Let the solution stand for 30 min. Aspirate the solution and dry the coverslips with a Kimwipe, by allowing the coverslips to dry naturally in air, or by blowing nitrogen on them. The amino-silanated coverslips remain viable for 48 hr. However, to prepare radial-gradient hydrogels, it is best to use the amino-silanated coverslips immediately after they are created to ensure uniform gel attachment. Prepare chloro-silanated glass slide(s). Using separate glass slides, spread about 100 μl of DCDMS onto each slide in the fume hood. Ensure that the solution coats the entire surface of the slides. Allow to react for up to 5 min before removing the excess DCDMS with a Kimwipe and rinse 1 min under distilled H₂O. Prepare statically compliant hydrogel(s). Mix acrylamide and bis-acrylamide to their desired concentrations in either distilled H₂O or PBS.

The elastic moduli will be slightly lower if the solutions are made in water, due to gel swelling when placed in cell culture media. This effect can be directly measured by AFM or other mechanical techniques. Acrylamide and bis-acrylamide can be kept together in solution for weeks to months, though the sterility of the stock solution should be closely maintained by filter sterilization. Degas the mixture under strong vacuum for 15 min to exhaust the solution of dissolved oxygen. Dissolved oxygen in the solution will act as a sink for the subsequent free radical polymerization. Degassing the solution not only speeds up polymerization but ensures more uniform polymerization as well. Add 1/100 total volume of APS and 1/1000 total volume of TEMED to gel solutions. Vortex the polymerizing solution. Quickly pipet 25 μl of the gel solution onto the treated side of the chloro-silanated glass slide(s) and add the amino-silanated coverslip(s) with the treated side down. Allow the gel to polymerize for 5 to 30 min. Monitor the unused solution to determine when the solution is fully polymerized. Shorter polymerization times may result in insufficient polymerization of all available monomers and may cause the mechanical properties of the hydrogels to vary from the values noted here. Remove the bottom glass slide and discard. Place the top coverslip-gel composite in a 35-mm petri dish or 6-well plate in PBS or dH₂O depending on what was used to dilute the acrylamide. Make sure that the gel-coated side faces up. To remove unpolymerized acrylamide rinse twice, each time for 5 min in PBS or distilled H₂O depending on what was used to dilute the acrylamide. These hydrogels can be stored for long periods of time without losing any of their mechanical properties. To store them, immerse the hydrogels in water or PBS to keep them hydrated and store them at 4° C.

### EXAMPLE 2

### Thiolated-Hyaluronic Acid (HA) Hydrogel as a Dynamic Substrate

The detailed methods for the HA hydrogels have been published for type one in Young and Engler. “Hydrogels with time-dependent material properties enhance cardiomyocyte differentiation in vitro,” Biomaterials (2011). Briefly, Hyaluronic Acid (HA) was obtained from Calbiotech (CA) and thiolated using a cleavable, carbohydrate selective, sulfhydryl-reactive crosslinker, PDPH (3-[2-Pyridyldithio]propionyl hydrazide) (Thermo Scientific-Pierce), MES Buffer (Thermo Scientific-Pierce), DMSO (Sigma), EDC (1-ethyl-3-[3-dimentylaminopropyl] carbodiimide hydrochloride) (Sigma), and DTT (dithiothreitol, Sigma). Alternatively, thiolated HA of similar functionality was also obtained from Glycosan Biosystems (UT). Poly(ethylene glycol) diacrylate (PEGDA) of different molecular weight was used as a crosslinker (Mw˜3400 Da from Glycosan Biosystems, UT and Mw˜258, 700 and 2000 Da from Sigma). For protein attachment on gels, EDC, NHS (N-Succinylamide) (Sigma) and type I rat tail collagen (BD Biosciences) in HEPES buffer (Sigma) was used. Polyacrylamide (PA) hydrogels were prepared from cross-linker n,n′-methylene-bis-acrylamide and acrylamide monomers (Fisher Scientific), and the same protein was covalently attached using a photoactivating cross-linker, sulfo-SANPAH (Pierce).

To fluorescently label collagen or cells on the surface of the hydrogel for imaging purposes, primary monoclonal mouse type I collagen antibody (C2456, Sigma), alpha-actinin (A7811, Sigma), rhodamine-phallodin, Hoescht (33342, Sigma) and Alexa Flour 488 or 568 conjugated goat anti-mouse secondary antibody (Invitrogen) were used. S amples were mounted using Fluoromount-G (SouthernBiotech). For ELISA, secondary goat anti-mouse HRP-conjugated antibody (62-6520, Zymed) and 3,3′,5,5′-Tetramethylbenzidine (TMB, Sigma) were used.

To examine myocardial stiffness and subsequent use in cell studies, chicken embryos were obtained from McIntyre Poultry Farm (Lakeside, Calif.). For histological analysis, hearts were embedded in optimal cutting temperature (OCT) solution (TissueTek) and stained using phosphomolybdic acid (Electron Microscopy Sciences-EMS), sirius red in 0.1% saturated picric acid (EMS), and mounted with Cytoseal (Richard Allen Scientific). For cardiomyocyte isolation, tissue was digested using 0.05% trypsin-EDTA (Invitrogen) and purified using a 70 μm cell strainer (BD Falcon). Cells were stored in normal heart medium (89% MEM alpha: L-glutamine (+), ribo-/deoxyribo-nucelosides (−), Invitrogen; 10% fetal bovine serum, Hyclone; and 1% penicilin:streptomycin, Invitrogen).

Fermentation-derived HA (sodium salt) of intermediate molecular weight, e.g., 769 kD, was digested in order to obtain low molecular weight HA of Mw˜200 kD as previously described. Briefly, 1 mg/mL HA was dissolved in 37° C. water of pH 0.5 (adjusted by the addition of 10M HCl) and mixed at 130 rpm for 6 hrs. pH was then adjusted to 7.0 with 1M NaOH, dialyzed against water for 4 days (12 kD molecular weight cutoff), and centrifuged before the supernatant was lyophilizated. HA was dissolved in MES Buffer at 5-10 mg/mL. 25 μL of 20 mM PDPH in DMSO was added per 1 mL of HA solution. The reaction was carried out at room temperature for 30-60 minutes. 12.5 μL of 0.5 M EDC in MES buffer was added per 1 mL of HA solution. The solution was mixed and incubated at room temperature for 2 hours to overnight with mixing. The solution was centrifuged in order to remove any precipitate that formed during the reaction. Any non-reacted PDPH molecule was removed by dialysis or gel filtration. In order to reduce the disulfide bond, 0.5 mL of 23 mg/mL DTT in MES buffer was added per 1 mL of PDPH-modified HA and incubated for 30 min at room temp. The solution was dialyzed or gel filtered in order to remove any excess DTT. Samples were lyophilized, dissolved in D₂O at 1 mg/mL and analyzed via 1H nuclear magnetic resonance (NMR) spectroscopy (JEOL ECA 500) to assess thiol substitution.

To prepare HA hydrogels of the appropriate stiffness to mimic heart stiffening, 4.53% (w/v) PEGDA with Mw˜3400 Da (polydispersity index or PDI˜3) in DG PBS and 1.25% thiolated HA in DG PBS were separately mixed at 37° C. with gentle shaking for up to 30 minutes. For swelling experiments, PEGDA with Mw˜258 Da, 700 Da, and/or 2000 Da (PDI˜1) were also used in a similar fashion. To initiate polymerization, solutions were combined at a volume ratio of 1 PEGDA solution: 4 HA solution to yield a 1% HA/0.9% PEGDA hydrogel, and 50 μL of the solution was placed between adhesive, aminosilanated and non-adhesive hydroxylated glass coverslips and allowed to polymerize in a humidified 37° C. incubator for 1 hr. Hydrogels bound to the aminosilanated coverslip were rinsed and stored in DG PBS in a humidified 37° C. incubator until use. To attach protein to the surface, 20 mM EDC, 50 mM NHS and 150 μg/mL type I rat tail collagen were mixed in HEPES buffer and incubated with the hydrogels overnight. Polyacrylamide gels were prepared as described previously. Briefly, gel cross-linker n,n′-methylene-bisacrylamide and acrylamide monomer concentrations were varied in distilled water and polymerized between adhesive, aminosilanated and non-adhesive hydroxylated glass coverslips using 1/200 volume of 10% ammonium persulfate and 1/2000 volume of n,n,n′,n′-tetramethylethylenediamine. To attach protein to the PA hydrogel surface, 0.5 mg/ml sulfo-SANPAH (Pierce) in 50 mM HEPES pH 8.5.

### EXAMPLE 3

### Mimicking Fibrotic Stiffening Associated with Disease with Dynamic Methacrylated-Hyaluronic Acid (MeHA) Hydrogel

Genome-wide association studies have identified single nucleotide polymorphisms (SNPs) at the 9p21 gene locus as increasing the risk of coronary artery disease (CAD) and myocardial infarction susceptibility. Associations have implicated SNPs in enhancing smooth muscle cell proliferation and endothelial permeability but have not identified adverse effects in cardiomyocytes.

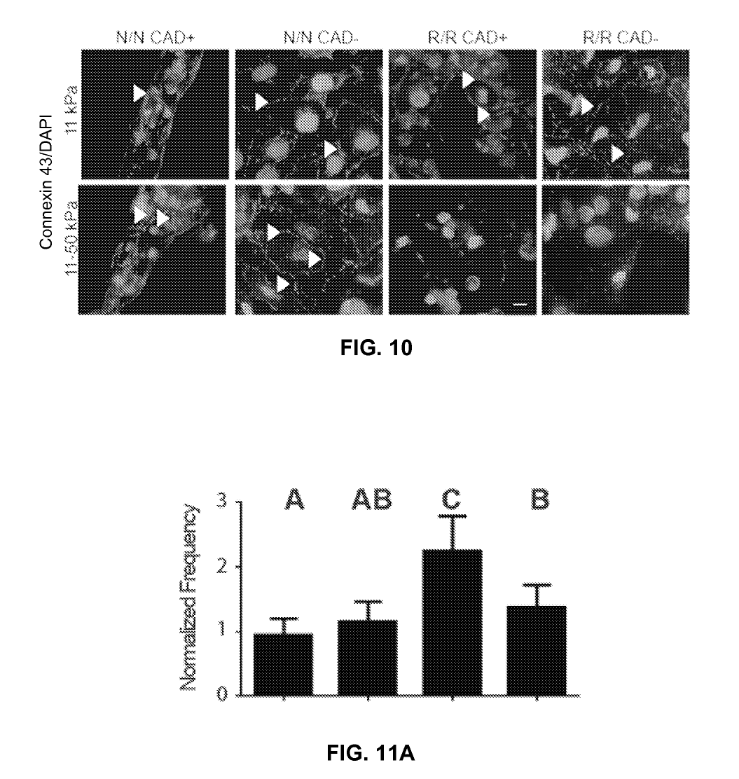

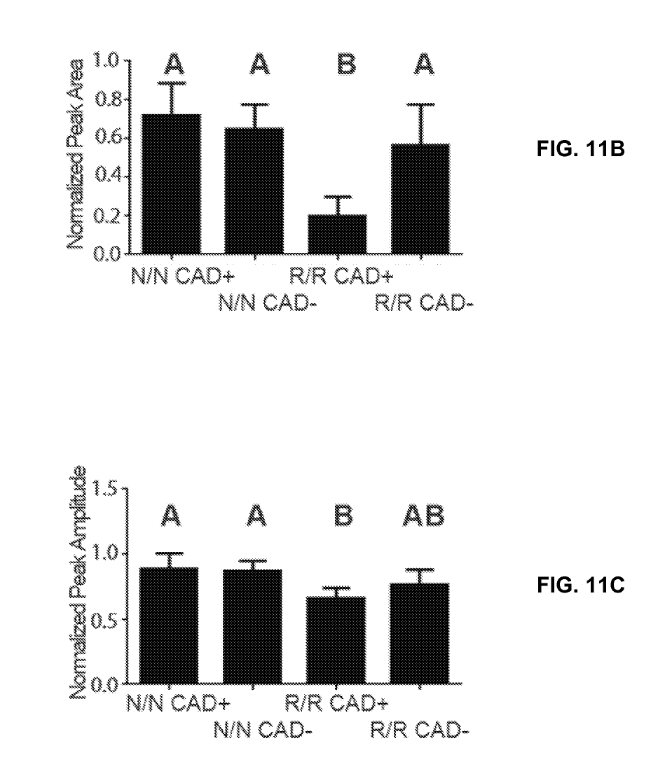

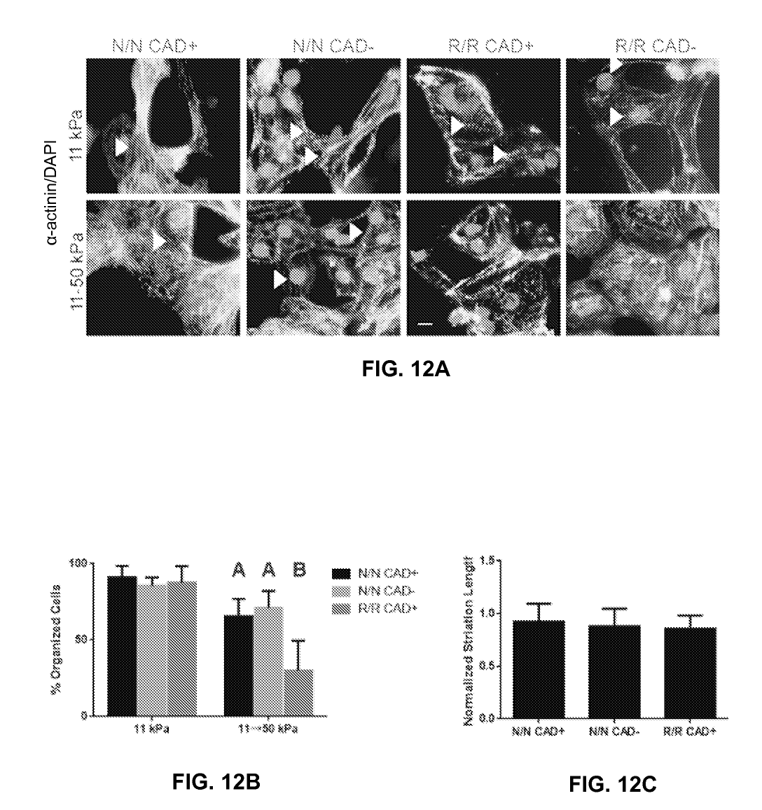

Using induced pluripotent stem cell-derived cardiomyocytes from patients that are homozygous risk/risk (R/R) and non-risk/non-risk (N/N) for 9p21 SNPs and either CAD positive (CAD+) or negative (CAD−), altered cardiomyocyte behavior was assessed when cultured on methacrylated-hyaluronic acid matrix (MeHA) capable of dynamically stiffening from healthy heart matrix stiffness, 11 kiloPascals (kPa), to that of fibrotic tissue, 50 kPa, to mimic the fibrotic stiffening associated with disease post-heart attack, i.e., “heart attack-in-a-dish.” (see, e.g., FIGS. 9-12). While all cardiomyocytes independent of genotype beat synchronously on soft matrices, R/R CAD+ cardiomyocytes cultured on dynamically stiffened hydrogels exhibited asynchronous contractions and had significantly lower correlation coefficients compared to N/N CAD+ or CAD− cardiomyocytes cultured under the same conditions. Furthermore, dynamic stiffening was associated with the loss of connexin 43 expression and gap junction assembly in R/R CAD+ cardiomyocytes, but not in N/N CAD+ or CAD− cardiomyocytes.

1% w/v sodium hyaluronate is reacted with 2.4 mL/gram methacrylic anhydride at pH 8 at 4° C. overnight, purified via dialysis, and lyophilized. ¹H NMR was used to confirm modification of hydroxyl groups (FIG. 4A). The resulting MeHA is mixed with phosphate buffered saline and triethanolamine Irgacure 2959 (photoinitiator) is added to the MeHA solution (0.1% w/v final concentration), vortexed, and pipetted between an aminosilane-coated and a chlorosilane-coated coverslip. Polymerizing a 3% w/v MeHA solution for 90 s using 350 nm UV (4.5 mW/cm²; FIG. 4B, step 1) yields a partially crosslinked 10 kPa hydrogel as measured by atomic force microscopy (AFM). MeHA hydrogels are then collagen-functionalized using 1-ethyl-3-(-3-dimethylaminopropyl) carbodiimide hydrochloride (EDC) and N-hydroxysuccinimide (NHS) chemistry to support cell adhesion and cells seeded on top of the hydrogel (FIG. 4B, step 2). With a second UV-activated free radical polymerization step, the hydrogel stiffens to at least 50 kPa (FIG. 4B, step 3; FIG. 4F).

These data are the first to demonstrate that specific heart disease niche changes can differentially affect cardiomyocyte function depending on 9p21 SNP status and induce a cardiac phenotype associated with 9p21 SNP status previously observed without the “heart attack-in-a-dish” model. It further suggests that this “heart attack-in-a-dish” model could be used throughout cell biology to understand disease phenotypes in vitro that require disease-like niche only after initially appearing normal, e.g., cancer.

All patents, patent applications and publications referred to in the present specification are also fully incorporated by reference.

Although the invention has been described with reference to the above example, it will be understood that modifications and variations are encompassed within the spirit and scope of the invention. Accordingly, the invention is limited only by the following claims.

1. A method of mimicking progression of human breast cancer in a hydrogel comprising:
(a) providing a methacrylated hyaluronic acid (MeHA) hydrogel having an elasticity defined by elastic constant E, wherein the MeHA hydrogel comprises a photoinitiator;
(b) exposing the MeHA hydrogel to UV radiation for sufficient time to achieve an elasticity of about 100 Pascal (Pa);
(c) seeding the MeHA hydrogel with an anchorage-dependent cell and allowing the cell to differentiate into a committed cell type; and
(d) thereafter exposing the MeHA hydrogel to additional photoinitiator and additional UV radiation.

2. The method of claim 1, wherein the MeHA hydrogel is overlayed with Matrigel after seeding and prior to differentiating.

3. The method of claim 1, wherein the MeHA hydrogel reaches an elasticity that exceeds 1000 Pa after the additional exposure to UV radiation.

4. The method of claim 1, wherein the MeHA hydrogel is a 1% w/v MeHA hydrogel.

5. The method of claim 1, wherein the anchorage-dependent cell is a mesenchymal stem cell, a human embryonic stem cell, or a human induced pluripotent stem cell that has been differentiated into a mammary epithelial cell.

6. The method of claim 5, wherein the committed cell type is a mammary epithelial cell.

7. The method of claim 1, wherein the photoinitiator is Irgacure 2959.

8. A method of mimicking progression of human heart disease or heart attack in a hydrogel comprising:
(a) providing a methacrylated hyaluronic acid (MeHA) hydrogel having an elasticity defined by elastic constant E, wherein the MeHA hydrogel comprises a photoinitiator;
(b) exposing the MeHA hydrogel to UV radiation for sufficient time to achieve an elasticity of about 10 kiloPascal (kPa);
(c) seeding the MeHA hydrogel with an anchorage-dependent cell and allowing the cell to differentiate into a cardiomyocyte, or seeding the MeHA hydrogel with a cardiomyocyte; and
(d) thereafter exposing the MeHA hydrogel to additional photoinitiator and additional UV radiation.

9. The method of claim 8, wherein the anchorage-dependent cell is a mesenchymal stem cell, a human embryonic stem cell, or a human induced pluripotent stem cell that has been differentiated into a cardiomyocyte.

10. The method of claim 8, wherein at day 2 after seeding, hypoxia is induced in the hydrogel.

11. The method of claim 10, wherein at day 5 after seeding, the additional UV radiation is irradiated onto the MeHA hydrogel to achieve an elasticity of at least 50 kPa.

12. The method of claim 8, wherein the MeHA hydrogel is a 4% w/v MeHA hydrogel.

13. The method of claim 8, wherein the photoinitiator is Irgacure 2959.

14. A method of mimicking progression of human heart disease or heart attack in a hydrogel comprising:
(a) providing a 4% w/v MeHA hydrogel having an elasticity defined by elastic constant E, wherein the MeHA hydrogel comprises a photoinitiator;
(b) exposing the MeHA hydrogel to UV radiation for sufficient time to achieve an elasticity of about 8-17 kiloPascal (kPa);
(c) seeding the MeHA hydrogel with an anchorage-dependent cell, allowing the cell to differentiate into a cardiomyocyte, and culturing the cardiomyocyte;
(d) inducing hypoxia in the culture at day 2 after culturing; and
(e) exposing the MeHA hydrogel to additional photoinitiator and additional UV radiation at day 5 after culturing to achieve an elasticity of at least 50 kPa in the MeHA hydrogel.

15. The method of claim 14, wherein the MeHA hydrogel is overlayed with Matrigel after seeding and prior to culturing.

16. The method of claim 14, wherein the anchorage-dependent cell is a mesenchymal stem cell, a human embryonic stem cell, or a human induced pluripotent stem cell that has been differentiated into a cardiomyocyte.

17. The method of claim 14, wherein the photoinitiator is Irgacure 2959.

18. A device for screening compounds for treating breast cancer in a subject comprising a solid substrate having disposed thereon a 1% w/v MeHA hydrogel having an elasticity defined by elastic constant E, wherein the MeHA hydrogel comprises a photoinitiator and the MeHA hydrogel is exposed to UV radiation for sufficient time to achieve an elasticity of about 100 Pascal (Pa), and an anchorage-dependent cell seeded within the MeHA hydrogel.

19. The device of claim 18 wherein the anchorage-dependent cell is a mesenchymal stem cell, a human embryonic stem cell, or a human induced pluripotent stem cell.

20. The device of claim 18, wherein the anchorage-dependent cell is allowed to differentiate into a mammary epithelial cell.

21. The device of claim 18, wherein the photoinitiator is Irgacure 2959.

22. A device for screening compounds for treating heart disease or heart attack comprising a solid substrate having disposed thereon a 4% w/v MeHA hydrogel having an elasticity defined by elastic constant E, wherein the MeHA hydrogel comprises a photoinitiator and the MeHA hydrogel is exposed to UV radiation for sufficient time to achieve an elasticity of about 8-17 kiloPascal (kPa), and an anchorage-dependent cell seeded within the MeHA hydrogel.

23. The device of claim 22, wherein the anchorage-dependent cell is a mesenchymal stem cell, a human embryonic stem cell, or a human induced pluripotent stem cell.

24. The device of claim 23, wherein the anchorage-dependent cell is allowed to differentiate into a cardiomyocyte.

25. The device of claim 22, wherein the photoinitiator is Irgacure 2959.

26-32. (canceled)
